(19) United States
(12) Patent Application Publication
(10) Pub. No.: US 2026/0194531 A1
(43) Pub. Date: Jul. 9, 2026

(54) METHODS FOR DETERMINING THE AMOUNT OF ENCAPSULATED AND FREE IRON IN A SAMPLE

(71) Applicant: SLOIRON, INC., Walnut Creek, CA (US)

(72) Inventor: Vincent P. Hackel, Walnut Creek, CA (US)

(73) Assignee: SLOIRON, INC., Walnut Creek, CA (US)

(21) Appl. No.: 19/010,844

(22) Filed: Jan. 6, 2025

(51) Int. Cl. G01N 33/68; B01D 61/18; G01N 1/40; G01N 33/53

(52) U.S. Cl. CPC G01N 33/683; B01D 61/18; G01N 1/4005; G01N 33/53; B01D 2239/1291; B01D 2311/10; G01N 2474/10

(57) ABSTRACT

This invention provides methods for quantitatively determining the concentration of free iron and ferritin-bound iron in a sample. The method includes diluting a ferritin-containing sample with a diluent to create a slurry, processing the slurry through a crossflow filtration system equipped with a membrane of at least 500 kilodaltons molecular weight cutoff, collecting the permeate containing free iron and the retentate containing ferritin-bound iron analyzing the iron concentrations in the permeate and retentate using a suitable analytical technique and calculating the mass balance of the total iron content. The invention also provides an antibody for use in quantitating the amount of ferritin in a sample and methods of treating iron deficiency in a patient in need thereof.

### CROSS REFERENCE TO RELATED APPLICATIONS

This application claims the benefit of U.S. Provisional Patent Application No. 63/619,566, filed on Feb. 29, 2024, the contents of which are incorporated herein by reference in their entirety.

### BACKGROUND

In humans and other animals, iron is essential for the implementation and maintenance of many vital cellular functions and biosynthetic processes, including oxygen transport, aerobic cellular activity, intracellular electron transport, and integral enzymatic reactions within body tissue. Iron deficiency is the most common nutritional deficiency worldwide, affecting 30 million people in both developed and developing countries. Iron deficiency has many repercussions, including diminished growth and learning in children.

Most stored iron in body tissues are contained in ferritin. Ferritin is an intracellular, protein-iron complex, formed from self-assembling subunits. The protein cage can reversibly form iron into a caged biomineral, Fe₂O₃·H₂O, in plants, animals, and bacteria. Iron oxy-biominerals inside the protein nanocages are iron concentrates for protein synthesis, and Fe(II)/oxygen/peroxide traps (Fenton chemistry reactants) for antioxidant protection. Ferritin concentrates iron 100 billion times above the solubility of ferric ion in a nontoxic, accessible form. Ferritin protein subunits, four α-helix bundles, contain a catalytic center that converts two Fe(II) atoms to an Fe(III)-oxo bridged dimer intermediate in mineralization. Ferritin proteins, including phytoferritin proteins, are composed of twenty-four identical or homologous subunits that assemble into a large spherical cage with a hollow cavity. In nature, phytoferritin proteins contain a range of 1000-2700 iron atoms per ferritin protein cage. However, each cage can accommodate up to about 4500 iron atoms. The two classes of ferritins are: i) maxi-ferritins, 24-polypeptide, 4-bundle subunit assemblies found in animals, plants, and bacteria; and ii) mini-ferritins (also called Dps proteins), 12-polypeptide, 4-bundle subunit assemblies in archaea and bacteria.

In animals, ferritin is present in tissues, especially in the liver, kidney, spleen, and bone marrow erythroid cells where it serves as an iron reserve for the production of hemoglobin. A small fraction of ferritin is in the serum and contributes little to overall iron storage but is used clinically as a reporter of iron levels in an animal. Ferritins occur in animals as approximately 25 distinct isoforms depending on their proportions of the two primary subtypes of ferritins, H or L. These distinct subtypes differ in their tissue distribution, rates and mechanisms of iron oxidation, core formation and physiological iron turnover.

Ferritin derived from plants and animals can be used as a dietary source for humans and other animals. Ferritin, which survives digestion largely intact, is more efficiently absorbed by the intestine than any other dietary iron source or iron supplement, because of the large amount of iron per ferritin molecule. Ferritin also survives treatment with high heat. Ferritin protein makes ferritin iron a naturally enteric coated, slow release, efficiently absorbed iron source. As such, ferritin can be used to supplement iron in animals in need of increased iron in their diet.

Previously, Western Blot analysis was needed to quantify the amount of ferritin present in a sample. This process is difficult, labor intensive, and not accurate. Described herein is a method for quantifying free iron as well as quantifying iron encapsulated in ferritin protein using capillary electrophoresis technology and an anti-ferritin antibody. The method is a significant improvement over previous techniques, allowing for sensitive and accurate quantification of ferritin.

### SUMMARY

One embodiment of the invention is directed towards methods for quantitatively determining the concentration of free iron and ferritin-bound iron in a sample, comprising diluting a ferritin-containing sample with a diluent to create a slurry, processing the slurry through a crossflow filtration system equipped with a membrane of at least 500 kilodaltons (kDa) molecular weight cutoff, collecting the permeate containing free iron and the retentate containing ferritin-bound iron, analyzing the iron concentrations in the permeate and retentate using a suitable analytical technique, and calculating the mass balance of the total iron content. In one aspect, the ferritin-containing sample is diluted with water in a ratio of 1:5 to 1:10 to create the slurry. In another aspect, the filtration is conducted at a backpressure of 15 psi and a flow rate of at least 1,500 mL/hr. In another aspect, the iron concentration in the permeate and retentate is analyzed using atomic absorption spectroscopy or inductively coupled plasma spectroscopy. Another aspect comprises monitoring the permeate flow rate using a scale under the sample tank to detect membrane fouling. In one aspect, the filtration system includes an automatic flushing mechanism to reduce membrane fouling. In one aspect, the analytical technique used for iron concentration analysis is colorimetric using a ferrozine-based assay. The invention also can include pre-treating the ferritin-containing sample with a chelating agent to stabilize free iron during filtration. In one aspect, the slurry is maintained at a controlled temperature between 4° C. and 25° C. during filtration to preserve protein integrity. In another aspect, an ultrafiltration step is done prior to crossflow filtration to remove impurities smaller than 10 kDa.

In another embodiment of the invention, a crossflow filtration system for separating free iron from ferritin-bound iron is described. The system comprises a sample tank for holding a slurry of ferritin-containing material, a crossflow filter with a 500 kDa molecular weight cutoff membrane, a pump configured to recirculate the slurry through the filter at a flow rate of at least 1,500 mL/hr, a backpressure regulator to maintain a transmembrane pressure of 15 psi, a permeate receiving vessel for collecting free iron, and means for monitoring and analyzing the iron content in the permeate and retentate. In one aspect, the membrane is made of polysulfone, polyethersulfone, or another biocompatible material. In another aspect, the system includes an integrated spectrophotometer for real-time analysis of iron concentrations in the permeate. In one aspect, the sample tank is equipped with an agitator to prevent settling of ferritin-containing material. In another aspect an automated system is used for continuous permeate and retentate collection. In another aspect, a temperature regulation unit can be used to maintain optimal processing conditions.

In another embodiment of the invention, a kit for determining ferritin-bound iron in a sample is described. The kit comprises a crossflow filtration membrane with a molecular weight cutoff of at least 500 kDa, instructions for diluting a ferritin-containing sample and processing it through the filtration system, components for collecting permeate and retentate samples, reagents or tools for determining iron concentration in the permeate and retentate and a guide for performing mass balance calculations to determine free and ferritin-bound iron. In one aspect, standards for the analytical technique used to quantify iron concentration are included in the kit, the pre-measured ferritin calibration standards can be derived from animal or plant sources. In another aspect, the reagents include a pre-prepared buffer solution optimized for ferritin stability. In another aspect, the kit can include disposable filters for one-time use in laboratory or field applications. In one aspect, the analytical tool is a portable spectrometer designed for on-site iron analysis.

In another embodiment of the invention, a method of producing a polyclonal antibody specific to soybean ferritin heavy chain (FTH1) protein is described. The method includes synthesizing a peptide spanning amino acids 109-136 of the FTH1 protein, immunizing a rabbit with the peptide, and affinity-purifying the resulting polyclonal antibody. In one aspect, the polyclonal antibody can be used in an immunoassay such as an enzyme-linked immunosorbent assay (ELISA) or Western blot to quantitate or detect the amount of ferritin in a sample, such as, for example, a sample is from an animal or a plant.

Another embodiment of the invention includes methods for determining ferritin concentration in a sample. The method comprises performing capillary electrophoresis on the sample using an anti-ferritin antibody, generating a standard curve based on known ferritin concentrations, comparing the electrophoresis results of the sample with the standard curve and calculating the ferritin concentration in the sample based on the comparison.

A further embodiment includes a kit for determining ferritin concentration in a sample. The kit contains an anti-ferritin antibody specific to ferritin, reagents for capillary electrophoresis, including buffers and standards, a software package for generating a standard curve and calculating ferritin concentrations.

An additional embodiment of the invention is a method for generating a standard curve for ferritin quantification. The method involves spiking samples with known concentrations of ferritin, performing capillary electrophoresis with an anti-ferritin antibody, plotting the detected signal intensity against ferritin concentration, and deriving a linear regression equation to calculate unknown ferritin concentrations.

Another embodiment of the invention is a method for determining ferritin concentration in a sample using a standard curve, comprising: performing capillary electrophoresis on the sample using an anti-ferritin antibody, comparing the detected ferritin peaks to the standard curve generated from known ferritin concentrations and calculating the ferritin concentration in the sample based on the comparison.

A further embodiment includes methods for mineralizing ferritin protein with iron atoms. The method includes obtaining ferritin protein containing less than the maximum amount of iron, adding iron to the ferritin protein under conditions that allow the protein cage to incorporate the added iron and isolating the mineralized ferritin protein. In one aspect, the iron atoms comprise ferrous sulfate, ferrous fumarate, ferrous gluconate, ferrous bisglycinate, or any other iron chelate or iron salt, or naturally occurring spring water that is high in iron. In another aspect, the starting ferritin protein is partially or fully purified ferritin protein. The mineralized ferritin protein can be used in methods of treatment or prevention of iron deficiency. The mineralized ferritin protein can be added into products for human or animal nutrition, such as food, beverages, nutraceuticals and supplements. Additionally, the mineralized ferritin protein can be added to a heat-processed substance such as food, beverages, nutraceuticals and supplements to increase the iron content of the heat-processed substance either before or after the substance is heat-processed. In one aspect, the conditions for mineralizing ferritin include maintaining a pH between 6.5 and 7.5. In an additional aspect, the ferritin protein is mineralized with a mixture of ferrous and ferric iron to mimic natural iron loading. Furthermore, the mineralized ferritin is lyophilized for long-term storage.

A further embodiment of the invention includes methods of treating iron deficiency in a patient, comprising administering an iron supplement derived from ferritin, wherein the supplement provides an iron intake of at least 5 mg/day for a period of at least 5 weeks, resulting in a significant improvement in hemoglobin levels and red blood cell quality, as measured by mean corpuscular hemoglobin concentration (MCHC). In one aspect, the iron intake is at least 10 mg/day for a period of at least 9 weeks. In another aspect, the ferritin-derived iron supplement is administered in a sustained-release formulation. In a further aspect, co-administration of ascorbic acid to enhance iron absorption is contemplated.

### DRAWINGS

These and other features, aspects, and advantages of the present invention will become better understood with regard to the following description, appended claims, and accompanying drawings where:

FIG. 1 depicts a standard curve graph of horse anti-ferritin antibody (13 exposure and 12 exposure lines) and the soy anti-ferritin antibody (2 exposure and 3 exposure lines) using identical samples of soy ferritin;

FIG. 2 depicts the standard curve from the data from the capillary electrophoresis from Table 4;

FIG. 3 depicts the standard curve obtained from the WES system showing generation of the horse ferritin standard curve;

FIG. 4 depicts a graph from the peak molecular weights for the standard curve which correspond to monomer, dimer and trimers of denatured ferritin subunits;

FIG. 5 depicts WES system graph from the first extraction;

FIG. 6 depicts WES system graph from the second extraction;

FIG. 7 depicts the WES system data from the first and the second water extraction;

FIG. 8 depicts the study schedule and menstrual cycle of the subjects;

FIG. 9 depicts a flow diagram of the progress from screening to analysis;

FIG. 10 depicts a graph of the changes in the serum ferritin levels for each subject before and after intake of trial supplements; and

FIG. 11 depicts the graphs showing five and nine weeks of intake.

### DETAILED DESCRIPTION

As used herein, the following terms and variations thereof have the meanings given below, unless a different meaning is clearly intended by the context in which such term is used.

The terms “a,” “an,” and “the” and similar referents used herein are to be construed to cover both the singular and the plural unless their usage in context indicates otherwise.

The term “buffer” or “buffered solution” refers to a mixture of acid and base which, when present in a solution, reduces or modulates changes in pH that would otherwise occur in the solution when an acid or base is added.

As used herein, the term “comprise” and variations of the term, such as “comprising” and “comprises,” are not intended to exclude other additives, components, integers or steps.

The term “ferritin” refers to the protein with caged iron oxide mineral that confers highly efficient intestinal iron absorption in living organisms and has biological and/or chemical activity and structure the same as, or substantially similar to, a natural, iron-containing ferritin. As such, ferritin includes a naturally occurring ferritin protein with iron mineral or a recombinant, iron mineral-reconstituted ferritin protein, comprising 12 or 24 ferritin subunits, wherein the subunits associate to form a spherical nanocage. Natural ferritins include animal ferritin including human ferritin, phytoferritin (plant ferritin) (derived from soybeans, and any other legume or legume process stream for example), microbial ferritins: mycoferritin (derived from fungi), or bacterial ferritin (bacterioferritin) or archaeal ferritin. Ferritin protein includes recombinant ferritin expressed by genetically-transformed microorganisms such as Escherichia coli, and other bacteria and yeasts. Ferritin expressed by genetically-transformed or recombinant microorganisms can have an amino acid sequence identical or analogous to natural ferritin. The term ferritin protein can include protein cages consisting of one or both animal isoforms, H and L or plant isoforms.

A “ferritin protein subunit” is defined as one of the 12 or 24 polypeptide subunits that make up a ferritin protein. The numbering system used herein for the identification of amino acids within ferritin subunits is based on the original sequence of horse spleen L ferritin (Swiss Protein Database Accession Number P02791). The horse spleen numbering system can be easily converted to a numbering system based on the human H sequence (Swiss Protein Database accession number P02794; the human L sequence accession number is P02792), which has four additional amino acids at the N-terminus. The human H sequence numbering therefore adds 4 to the corresponding amino acid number in horse spleen ferritin. For example, L134 by horse spleen numbering corresponds to L138 by human H sequence numbering.

“Apoferritin” refers to the iron-free form of the protein, i.e., the protein in the unmineralized state.

A “ferritin pore” is one of the external or internal ferritin cage pores that lead to the eight Fe²⁺ exit/entry ion channels in an assembled ferritin protein cage; the channels and pores are formed by trimers of ferritin subunits. In an intact, 24 subunit ferritin protein cage, there are eight three-fold axes of symmetry, each at a junction of three ferritin subunits. Each ferritin pore and ion channel is formed by these three-way junctions of ferritin subunits. The pores can be visualized in crystals of ferritin proteins by X-ray crystallography and analyzed in solutions by changes in the rate of Fe²⁺ exit.

An “immunoblot” or “immunodetection” is a specific type of biochemical test that measures the presence or concentration of a protein (referred to as the “analyte”) in solutions that frequently contain a complex mixture of substances including other proteins. The methods and techniques involved in immunoassays are well known by those in the art.

“Isolation” or “isolation of ferritin” as used herein means separation of ferritin from other components in the plant or animal material, which provides a substantially pure target compound, such as a substantially pure ferritin.

Substantially pure ferritin contains ferritin in an amount of from about 5% to about 100%, from about 15% to about 80%, from about 50% to about 85%, from about 65% to about 95% by weight of the total protein in the material processed by the method of the invention.

The terms “individual,” “subject” and “patient” are used interchangeably herein, and generally refer to a mammal. The term “mammal” is defined as an individual belonging to the class Mammalia and includes, without limitation, humans, experimental animals such as, for example, rodents, domestic and farm animals, zoo, sports, and pet animals, such as for example, cows, sheep, dogs, horses, cats and cows.

A “legume” can be one or more soybeans, yellow peas, green peas, lentils, chickpeas (also called garbanzos), peanuts, trefoil, pinto beans, Great Northern beans, navy beans, red beans, black beans, dark or light red kidney beans, fava beans, baby lima beans, pink beans, mayocoba beans, small red beans, black-eyed peas (also called cow peas), cranberry beans, white beans, rice beans, butter beans, and combinations of any of the foregoing. The legume can be any of a variety of species, including, e.g., a Phaseolus species (e.g., Phaseolus vulgaris), a Pisum species (e.g., Pisum sativum), a Lens species (e.g., Lens vulgaris, Lens culinaris), a Cicera species (e.g., Cicera arietenum), a Vigna species (e.g., Vigna unguiculata), a Glycine species (e.g., Glycine max), and combinations of any thereof.

The term “nutraceutical formulation” refers to a food or part of a food that offers medical and/or health benefits including prevention or treatment of disease. Nutraceutical products range from isolated nutrients, dietary supplements, genetically engineered designer foods, functional foods, herbal products and processed foods such as cereal, soup and beverages. The term “functional foods,” refers to foods that include “any modified food or food ingredients that may provide a health benefit beyond the traditional nutrients it contains.” Nutraceutical formulations of interest include foods for veterinary or human use, including food bars (e.g. cereal bars, breakfast bars, energy bars, nutritional bars); chewing gums; drinks; fortified drinks; drink supplements (e.g., powders to be added to a drink); tablets; lozenges; candies; and the like.

The term “reconstituted,” “mineralized” or “remineralized” refers to the addition of iron atoms to ferritin protein.

The term “solution” refers to a composition comprising a solvent and a solute, and includes true solutions and suspensions. Examples of solutions include a solid, liquid or gas dissolved in a liquid and particulates or micelles suspended in a liquid.

A “supplement” or “dietary supplement” as used herein is useful for supplementing, replenishing, and increasing the iron supply to humans, animals and plants, and for treating various disorders and conditions. A dietary supplement can be formulated for oral administration. As contemplated in the present invention, a dietary supplement includes ferritin in an amount of from about 10% to about 90% by weight of the total protein in the supplement. For example, subject dietary supplement includes ferritin in an amount of from about 0.1%-9%, 10% to about 15%, from about 15% to about 20%, from about 20% to about 25%, from about 25% to about 30%, from about 30% to about 35%, from about 35% to about 40%, from about 40% to about 45%, from about 45% to about 50%, or from about 55% to about 90%, and from about 90% to about 100% by weight of the total protein in the supplement. For oral preparations, a subject dietary supplement can be formulated with appropriate additives to make tablets, powders, granules or capsules, gummies, liquids, fortified foods, snacks, bread, yogurt, ice cream, rice, etc., for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. A dietary supplement can be administered in one, or more than one doses per day. A dietary supplement can be administered at various frequencies, e.g., four times daily, three times daily, twice daily, once daily, every other day, three times per week, twice per week, or once per week.

A “therapeutic composition” as used herein means a substance that is intended to have a therapeutic effect such as, for example, a pharmaceutical composition, a nutraceutical, a dietary supplement, and other substances. A therapeutic composition may be configured to contain a pharmaceutically acceptable carrier. The therapeutic composition may contain pharmaceutically acceptable excipients, such as vehicles, adjuvants, carriers or diluents, as well as pharmaceutically acceptable auxiliary substances, such as pH adjusting and buffering agents, tonicity adjusting agents, stabilizers, and wetting agents.

As used herein, the phrases “therapeutically effective amount” and “prophylactically effective amount” refer to an amount that provides a therapeutic benefit in the treatment, prevention, or management of a disease or an overt symptom of the disease. The therapeutically effective amount may treat a disease or condition, a symptom of disease, or a predisposition toward a disease, with the purpose to cure, heal, alleviate, relieve, alter, remedy, ameliorate, improve, or affect the disease, the symptoms of disease, or the predisposition toward disease. The specific amount that is therapeutically effective can be readily determined by ordinary medical practitioner, and may vary depending on factors known in the art, such as, e.g. the type of disease, the patient's history and age, the stage of disease, and the administration of other therapeutic agents.

The terms “treatment” or “treating” as used herein covers any treatment of a condition treatable by iron in a living organism, for example, a human, and includes:
(i) preventing the condition from occurring in a subject which may be predisposed to the condition but has not yet been diagnosed as having it;
(ii) inhibiting the condition, e.g., arresting or slowing its development; or
(iii) relieving the condition, e.g., causing regression of the condition.

Ferritin is a form of dietary iron that contains a protein cage with iron oxide mineral inside the protein cage. In contrast with other iron supplements and dietary iron sources, ferritin's protein coat protects a user's intestine from free radical chemistry caused by iron and iron salts, which can irritate the intestine. In addition, ferritin iron is released slowly into the blood from the intestine, which also allows for protection of the intestine from free radical chemistry caused by iron.

Ferritin iron is absorbed by the intestine using a mechanism that is different from the mechanism of absorption of other iron sources such as non-heme iron supplements or heme iron from meat. Ferritin iron is absorbed in the intestine through a protein uptake pathway, rather than an ion pathway used by other iron supplements. Humans have been consuming ferritin iron for millennia in forms such as ferritin-rich legumes, which have been cultivated for 12,000 years. Contemporary cultures include legumes in their traditional and modern diets. In addition, phytoferritin can be eaten by vegans, which is important since many vegan diets are iron deficient and need to be supplemented with iron.

Phytoferritin contains an average of 1000 iron atoms per protein cage, and animal ferritin contains an average of 1500-2000 iron atoms per protein cage, which allows for more efficient absorption of ferritin iron by the body. In other words, for one transport event in the intestine, the user's body would obtain 1000 times as much iron as it would from non-heme iron salts and chelators.

Ferritin iron is well absorbed by animals. In a rat model, ferritin has been shown to cure iron deficiency. In humans, ferritin iron is absorbed on the order of 20-30%.

Plant material can be used as a starting material to isolate phytoferritin in a substantially pure form. A typical starting plant material is a legume such as a soybean. In addition to soybeans, yellow peas, green peas, lentils, chickpeas, peanuts, trefoil, pinto beans, Great Northern beans, navy beans, red beans, black beans, dark or light red kidney beans, fava beans, green baby lima beans, pink beans, mayocoba beans, small red beans, black-eyed peas, cranberry beans, white beans, rice beans, butter beans, or a combination thereof can be used as starting plant material.

The plant material used to isolate phytoferritin can include the whole plant, or any ferritin-rich portion of a plant, e.g., seed, stem, fruit, leaf, root (e.g., nodulating root), flower, stem, etc. In some cases, the source of the phytoferritin is one or more of a seed, a nodulating root, and a leaf. Where the source of the phytoferritin is a seed or a bean, the phytoferritin can be obtained from the whole seed or bean, or a part of a seed or bean, e.g., the hull.

The starting plant material can also be a processing stream or a waste stream resulting from the processing of soy or other beans. For example, the source of the isolated ferritin can be a waste stream from the production of tofu or soy milk from soybeans. Processing soy for soy milk produces an insoluble by-product of soy, called okara. Either wet or dried okara or other material from legume waste process streams can be used as starting plant material.

The plant material from the waste stream and/or the legumes themselves are treated to isolate the ferritin, followed by concentration of the ferritin. The concentrated ferritin can then be used to treat humans and other animals in need thereof, such as, for example, treatment of an iron deficiency.

Ferritin can be isolated from plant material using methods as those described in U.S. Pat. No. 8,476,061. To isolate ferritin from plant material, the plant material is separated into soluble and insoluble fractions. A neutral saline buffer is then added to the insoluble fraction to make an insoluble solution, which is clarified into soluble and insoluble solution fractions. The soluble solution is treated enzymatically with one or more glycosidase enzymes. The clarified soluble solution is fractionated to remove non-ferritin components and the isolated ferritin is concentrated.

Alternatively, the ferritin in the clarified soluble solution can be further purified prior to concentration. One such purification method includes the steps of 1) centrifugation of the clarified soluble solution, followed by 2) tangential flow or crossflow filtration of the clarified material across a membrane, and optional 3) concentration of the clarified, filtered material such as by the spray dry method. The resulting purified ferritin, either the clarified material after step 2, or the powder after step 3, is more water soluble than ferritin from the clarified soluble solution.

Another method to isolate ferritin from plant material includes the steps of: (a) separation of the plant material into soluble and insoluble fractions; (b) enzymatic removal of non-ferritin components from the soluble fraction with one or more glycosidase enzymes, and (c) concentration of the isolated ferritin from step (b).

Another method to isolate ferritin from plant material is the addition of a neutral saline buffer to plant material followed by enzymatic treatment with one or more glycosidase enzymes. The enzymatically treated plant material substrate is then separated and clarified into soluble and insoluble fractions, which are then fractionated to remove non-ferritin components. The isolated ferritin from the fractionated soluble solution is then concentrated or further purified as described above.

In animals, ferritin is present in high amounts in the liver, kidney, spleen, and bone marrow. Ferritin may be derived from the tissues of animals. Iron-containing ferritin derived from animals can be also used by humans and other animals that need increased iron in their diet. Ferritin can be isolated from animal material as described in US U.S. Pat. No. 8,476,061.

Isolated plant or animal ferritin is particularly useful because it can be added into products for human or animal nutrition, such as food, beverages, nutraceuticals and supplements. A therapeutic or supplementary composition containing isolated plant or animal ferritin can be delivered to an organism in need of iron. The organism can be an animal such as a mammal, including a human. Alternatively, the organism can be a plant.

The isolated ferritin agents and therapeutic compositions can be administered by continuous delivery, intermittent delivery, or through a combination of continuous and intermittent delivery. Many factors can influence the dosage and timing required to effectively treat a subject, including, but not limited to, the severity of the disease or disorder, previous treatments, the general health and/or age of the subject, and other diseases present. Moreover, treatment of a subject with a therapeutically effective amount of a composition can include a single treatment or a series of treatments. In addition to their administration individually or as a plurality, the therapeutic compositions of the invention can be administered in combination with other known agents effective in treatment of diseases. In any event, the administering physician can adjust the amount and timing of administration of the therapeutic composition on the basis of results observed using standard measures of efficacy known in the art or described herein.

In nature, phytoferritin contains a range of 1000-2700 iron atoms per ferritin protein cage. Ferritin proteins, including phytoferritin proteins, are composed of twenty-four identical or homologous subunits that assemble into a large spherical cage with a hollow cavity. The cage can accommodate up to about 4500 iron atoms.

The protein-caged iron mineral present in ferritin can be expanded in a ferritin protein containing less than the amount of iron possible in each of its protein cages or reconstituted in apoferritin. The starting ferritin protein (apoferritin or ferritin protein containing less than the maximum amount of iron) can be obtained by any means, including the ferritin protein isolation methods described above.

Using the calculation of the amount of ferritin iron contained in the starting solid material, a mineralization procedure can be done to incorporate additional iron atoms in each ferritin protein. The target of iron atom incorporation depends on the amount of iron atoms contained in the starting solid material. A target mineralization of between 2000-4500 iron atoms per ferritin cage is desirable.

The mineralization procedure is carried out as follows. First, iron atoms in the form of ferrous sulfate, ferrous fumarate, ferrous gluconate, ferrous bisglycinate, or any other iron chelate or iron salt, or naturally occurring spring water that is high in iron, is added to partially or fully purified or isolated ferritin protein, and the iron atoms are allowed to incorporate inside the cages of the ferritin protein.

After mineralization, the mineralized ferritin protein can be used in the various applications described above. For example, mineralized ferritin protein can be added into products for human or animal nutrition, such as food, beverages, nutraceuticals and supplements. Additionally, mineralized ferritin protein can be added to a heat-processed substance to increase the iron content of the heat-processed substance. A heat-processed substance can be food or a beverage. The isolated ferritin can be added to the heat-processed substance either before or after the substance is heat-processed. The mineralized ferritin can also be used in the methods of treatment or prevention of iron deficiency as described above.

Rough determination of the ferritin protein and iron concentration in plant extracts has been shown previously using quantitative Western blotting with a rabbit polyclonal anti-soybean ferritin antiserum, and/or pea ferritin antiserum, and/or horse ferritin antiserum. However, it is desirable to obtain an accurate quantitation of ferritin. The present invention provides methods to obtain an accurate quantitation of free iron, ferritin-bound iron and quantitation of ferritin protein in a sample.

### Example 1—Determination of Free Iron Versus Ferritin-Bound Iron in a Sample

Described herein is a method for quantitatively determining the concentration of free iron, which is iron that has not been encapsulated into a ferritin cage, versus ferritin-bound iron. The methods of the present invention contemplate an accurate quantitation of iron derived from plant and animal ferritin, either natural or from recombinant apoferritin reconstituted with iron mineral. With this process, free iron passes through a crossflow filter and mass balance calculations are done to determine the amount of free iron. Using cross flow filter technology in this manner any small non-bound iron atoms pass through the filter and can be quantified.

To determine the free iron present in isolated ferritin, two liters of ferritin isolated by any means were first diluted with eight liters of water, forming a slurry. The slurry was mixed, covered, for one hour in a sample tank.

Next, a crossflow system was briefly turned on (i.e. 2 minutes in this example) to mix the system's hold volume with the slurry sample, recirculating the slurry through the crossflow filter using a pump. A critical component to the crossflow system is the backpressure regulator controls. The system hold volume is critical, as detailed below. Once mixed, 500 mL was removed and frozen as starting material. This was used as Sample A and is the starting full iron amount in the mass balance Equation 1, shown below. This requires a valve to reduce the flow of the pump while increasing the back pressure across the filter membrane. There is also a pressure gauge on the backpressure controls to accurately measure this transmembrane pressure. In this system the operating back pressure was 15 psi.

$$A = B + C \qquad Equation 1$$

Crossflow filtration effectively separates free iron from ferritin-bound iron using size-selective membranes. The setup ensures precise quantification and minimizes sample loss. The setup can be any style, but should have sufficient capacity and crossflow to be able to achieve at least 1,500 ml/hr. In this example, a 500 kilodalton (kDa) crossflow filter unit was used. The system used in this example was a Romicon HF UF Cartridge 1018-0.7-106-PM500 PN: 0720199 with a Simer 1 HP 9.8 AMP pump, model 2825ss-01. This is a centripetal pump with 10 gallons per minute (gpm) at zero feet of head pressure.

The system hold volume should be known, as this will be added diluent in the overall volume used to dilute the two liters of starting slurry. The system used in this example contained one liter hold volume. The permeate was collected in a permeate receiving vessel. A scale is used under the sample tank. This allows for monitoring of the permeate flow rate, as the weight in the sample tank decreasing over time is due to the permeate crossing the membrane filter. This monitoring is important in order to determine when the membrane begins to foul.

The system ran until 3 liters of permeate was collected in the receiving vessel. The system was stopped, the permeate (Sample B, free iron) and retentate (Sample C, ferritin-bound iron) samples and final volumes were recorded. Fresh water was used to flush the system and discard material in the system. This lost volume is considered in calculations.

500 ml of permeate Sample B and 500 ml of retentate Sample C was packaged and frozen for future testing. Any remaining material was also packaged and saved as needed for future use.

$$A = \begin{matrix}2 L finished \\ product\end{matrix} + \begin{matrix}8 L \\ water\end{matrix} + \begin{matrix}1 L hold \\ volume\end{matrix} - \begin{matrix}0.5 L sample \\ A\end{matrix}$$

$$B = 3 L permeate$$

$$C = 6.5 L retentate$$

$$1 L hold volume of system remaining$$

The mass balance of iron content is done once the test results are obtained. Basic mass balance techniques are used to calculate the iron content of the permeate and retentate. The samples are analyzed for iron concentration using an appropriate analytical technique, such as atomic absorption spectroscopy (AAS), inductively coupled plasma (ICP), or other suitable methods as described further below. The present invention can include components required to determine the amount of ferritin iron in a sample, provided separately or in a kit.

### Example 2—Production of Custom Rabbit Ferritin Polyclonal Antibody

Custom rabbit anti-ferritin polyclonal antibodies were produced at GenScript (Piscataway, NJ) using a peptide-KLH conjugate antigen having the amino acid sequence spanning amino acids 109-136 of the soybean ferritin heavy chain (FTH1) protein (UniProtKB P19976). The polyclonal anti-ferritin antibody was affinity-purified. For long-term storage, the polyclonal antibody was aliquoted and stored at −20° C. or below, avoiding repeated freezing and thawing cycles.

An ELISA was performed using the 109-136 amino acid soybean FTH1 antigen as the coating antigen at a concentration of 4 μg/ml, 100 μl/well. The coating buffer used was Phosphate Buffered Saline (PBS, pH 7.4) with 0.02% sodium azide. The purified anti-ferritin antibodies were used as the primary antibody. The secondary antibody used was an HRP conjugated goat anti-rabbit IgG (H+L). The results are shown in Table 1 below.

TABLE 1
ELISA results for pre-immune serum and purified antibody

| | | Purified anti-ferritin |
|---|---|---|
| | Dilution | antibody (A450 nm) |
| NC | 1:1,000 | 0.051 |
| 1 | 1:1,000 | 3.448 |
| 2 | 1:2,000 | 3.431 |
| 3 | 1:4,000 | 3.080 |
| 4 | 1:8,000 | 2.597 |
| 5 | 1:16,000 | 2.401 |
| 6 | 1:32,000 | 1.889 |
| 7 | 1:64,000 | 1.308 |
| 8 | 1:128,000 | 0.909 |
| 9 | 1:256,000 | 0.501 |
| 10 | 1:512,000 | 0.298 |
| 11 | Blank | 0.053 |
| 12 | Blank | 0.053 |
| | Titer: | 1:512,000 |

Starting dilution: 1:1,000 (Equivalent to 1 μg/ml)
The titer is the highest dilution with S/B (Sample/Blank) >=2.1
NC is negative control (Pre-immune serum)

An indirect ELISA was also performed using the peptide of SEQ ID NO. 1 as the coating antigen at a concentration of 4 μg/ml, 100 μl/well. The coating buffer used was PBS (pH 7.4) with 0.02% sodium azide. The secondary antibody used was an HRP conjugated anti-rabbit IgG Fc monoclonal secondary antibody (GenScript, Piscataway, NJ, Cat. No. A01856). The results of two anti-ferritin antibody batches are shown in Table 2 below.

TABLE 2
ELISA results of affinity-purified antibody

| | | | | | | |
|---|---|---|---|---|---|---|
| Concen- | 1000 | 500 | 250 | 125 | 62.50 | 31.25 |
| tration | | | | | | |
| (ng/ml) | | | | | | |
| Sample\ | 1:1000 | 1:2000 | 1:4000 | 1:8000 | 1:16,000 | 1:32,000 |
| Dilution | | | | | | |
| Antibody | 2.749 | 2.763 | 2.690 | 2.565 | 2.470 | 2.110 |
| Batch | | | | | | |
| #3942 | | | | | | |
| Antibody | 2.785 | 2.776 | 2.717 | 2.612 | 2.355 | 2.133 |
| Batch | | | | | | |
| #3944 | | | | | | |

| | | | | | | |
|---|---|---|---|---|---|---|
| Concen- | 15.62 | 7.81 | 3.90 | 1.95 | Blank | |
| tration | | | | | | |
| (ng/ml) | | | | | | |
| Sample\ | 1:64,000 | 1:128,000 | 1:256,000 | 1:512,000 | Blank | Titer |
| Dilution | | | | | | |
| Antibody | 1.708 | 1.150 | 0.698 | 0.455 | 0.060 | 1:512,000 |
| Batch | | | | | | |
| #3942 | | | | | | |
| Antibody | 1.642 | 1.230 | 0.743 | 0.416 | 0.055 | 1:512,000 |
| Batch | | | | | | |
| #3944 | | | | | | |

The titer is the highest dilution with S/B (Signal/Blank) >= 2.1, the OD₄₅₀ in blank is the average of two technical replicates.
The starting concentration of 1 mg/ml and the corresponding dilution ratio is calculated based on the actual concentration.

Indirect ELISA results of pre-immune serum and affinity-purified antibody after the third immunization was also performed using the peptide of SEQ ID NO. 1 as the coating antigen at a concentration of 4 μg/ml, 100 μl/well. The coating buffer used was PBS (pH 7.4) with 0.02% sodium azide. The secondary antibody used was HRP conjugated anti-rabbit IgG secondary antibody. The results are shown in Table 3 below.

TABLE 3
ELISA results of pre-immune serum and affinity-
purified antibody after the 3^{rd} immunization

| | | | | | | | |
|---|---|---|---|---|---|---|---|
| Concen- | NC | 1000 | 500 | 250 | 125 | 62.5 | 31.25 |
| tration | | | | | | | |
| (ng/ml) | | | | | | | |
| Sample\ | 1:1000 | 1:1000 | 1:2000 | 1:4000 | 1:8000 | 1:16,000 | 1:32,000 |
| Dilution | | | | | | | |
| Antibody | 0.085 | 2.664 | 2.641 | 2.639 | 2.483 | 2.344 | 2.106 |
| Batch | | | | | | | |
| #3942 | | | | | | | |
| Antibody | 0.067 | 2.809 | 2.731 | 2.684 | 2.618 | 2.511 | 2.340 |
| Batch | | | | | | | |
| #3944 | | | | | | | |

| | | | | | | |
|---|---|---|---|---|---|---|
| Concen- | 15.62 | 7.81 | 3.90 | 1.95 | Blank | / |
| tration | | | | | | |
| (ng/ml) | | | | | | |
| Sample\ | 1:64,000 | 1:128,000 | 1:256,000 | 1:512,000 | Blank | Titer |
| Dilution | | | | | | |
| Antibody | 1.749 | 1.276 | 0.843 | 0.517 | 0.061 | >1:512,000 |
| Batch | | | | | | |
| #3942 | | | | | | |
| Antibody | 2.054 | 1.654 | 1.186 | 0.762 | 0.068 | >1:512,000 |
| Batch | | | | | | |
| #3944 | | | | | | |

The titer is the highest dilution with S/B >= 2.1, the OD₄₅₀ in blank is the average of two technical replicates.
The starting concentration of 1 mg/ml and the corresponding dilution ratio is calculated based on the actual concentration.
NC is negative control (pre-immune serum)

### Example 3—Determination of Ferritin Concentration in a Sample

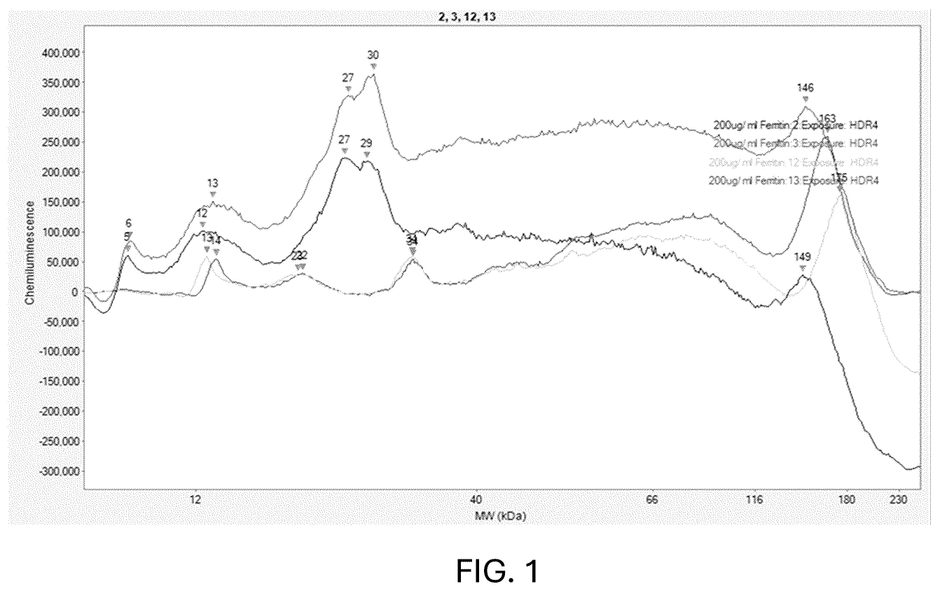

The present invention contemplates a method for determining the amount of ferritin iron in a sample. As shown in FIG. 1, a capillary electrophoresis was done using the anti-ferritin antibody described in Example 2. The capillary electrophoresis was done using the automated WES system (Western Blot Service, Austin, TX). The WES system uses capillary electrophoresis with automated protein fractionation, immobilization and immunodetection to quantify protein in the sample.

Using the anti-ferritin antibody, differences between horse and legume ferritin sources were seen. The results of horse anti-ferritin antibody (13 exposure and 12 exposure lines) and the soy anti-ferritin antibody described in Example 2 (2 exposure and 3 exposure lines) using identical samples of soy ferritin are shown in FIG. 1.

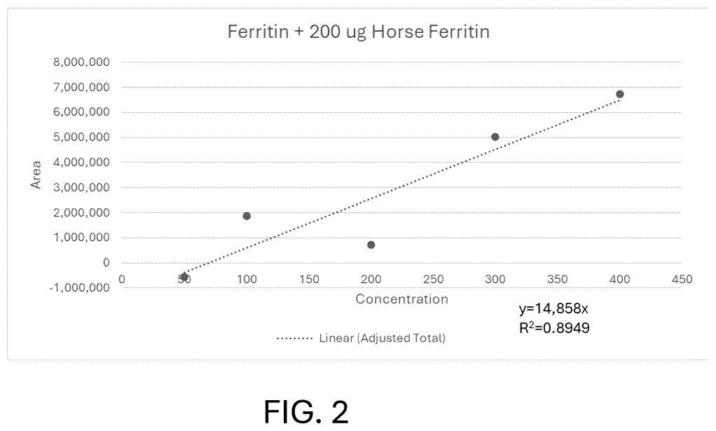

The linearity of the concentration and fit were used to generate a standard curve from the data from the capillary electrophoresis, as can be seen in Table 4 and FIG. 2, which show samples spiked with 200 μg horse ferritin and isolated soy ferritin. The standard curve can be used to determine the amount of ferritin in a sample. The specificity of the specific antibody for ferritin and the accuracy of the capillary electrophoresis process with this antibody is a significant improvement over previous techniques used to quantify the amount of ferritin in a sample.

TABLE 4
Ferritin concentration

| | Ferritin + | | | | |
|---|---|---|---|---|---|
| | 200 ug | | | | |
| Ferritin | Horse | | | | |
| Sample | Ferritin | | Adjusted | | |
| (ug/mL) | (ug/ml) | Total | Total | 27 kDa | 29 kDa |
| 400 | 600 | 12,790,028 | 6,725,028 | 5,679,493 | 7,110,535 |
| 300 | 500 | 11,079,156 | 5,014,156 | 5,377,002 | 5,702,153 |
| 200 | 400 | 6,779,485 | 714,485 | 5,390,932 | 1,388,554 |
| 100 | 300 | 7,918,614 | 1,853,614 | 4,885,142 | 3,033,472 |
| 50 | 250 | 5,506,088 | −558,912 | 4,756,741 | 749,348 |

Equation from all Horse Ferritin y = 30,325x
x = 200
y = 6,065,000
Horse Ferritin offset 6,065,000

### Example 4—Ferritin Concentration Standard Curve

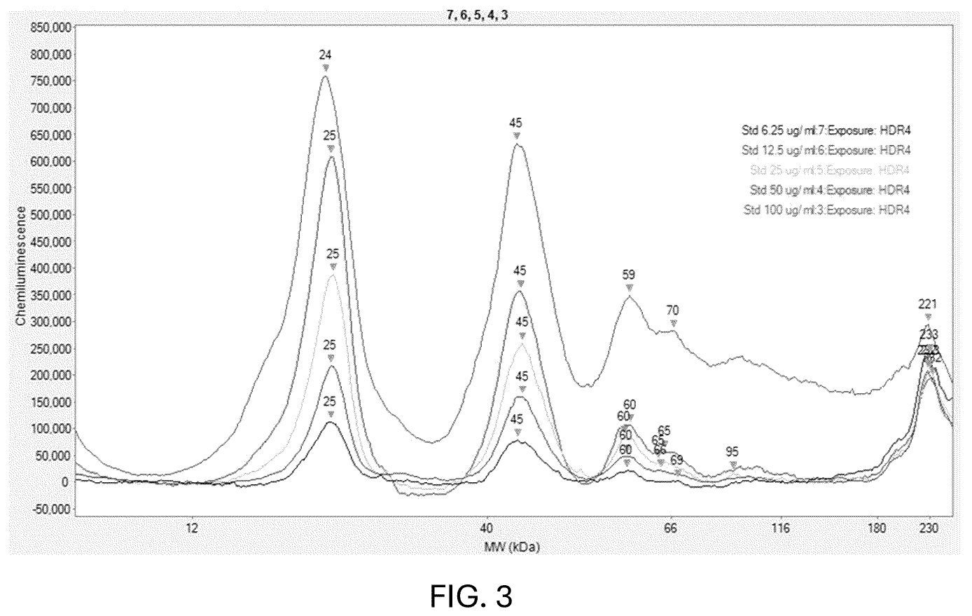

Capillary electrophoresis using the WES system was performed in order to develop a standard curve for determining the ferritin concentration in a sample using the ferritin antibody described in Example 2. FIG. 3 depicts the image obtained from the WES system showing generation of the horse ferritin standard curve.

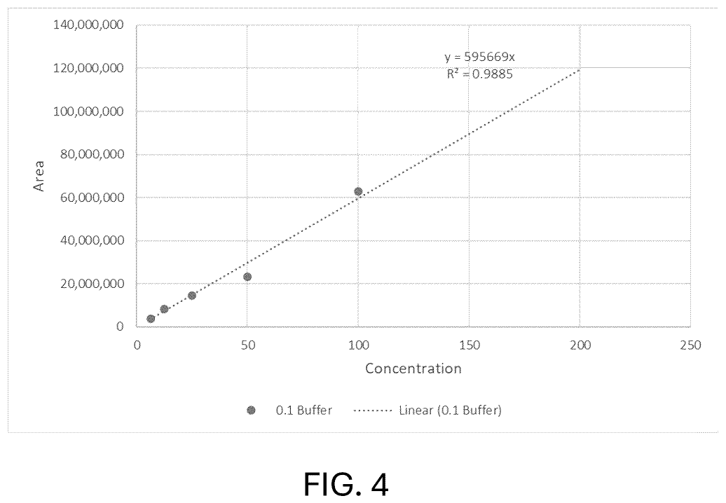

Table 5 shows the numerical values of the areas under the peaks and the peak molecular weights for the standard curve. These correspond with monomer, dimer and trimers respectively for denatured ferritin subunits. The linearity is very good with an R2 value of 0.9885 as shown in FIG. 4.

TABLE 5
Ferritin standard curve
Standard Curve Data
Horse 0.1 Buffer 95 for 5 min.

| Sample | | | | | |
|---|---|---|---|---|---|
| ug/ml | Total | 24 kDa | 45 kDa | 60 kDa | 66 kDa |
| 200 | | 22,794,767 | 14,167,256 | 3,593,994 | 3,903,354 |
| 100 | 62,832,396 | 24,282,995 | 19,504,810 | 3,062,747 | 15,981,844 |
| 50 | 23,174,393 | 12,817,600 | 7,758,192 | 1,365,322 | 1,233,279 |
| 25 | 14,671,736 | 7,379,095 | 5,532,580 | 1,132,494 | 627,567 |
| 12.5 | 8,157,211 | 3,771,931 | 3,334,991 | 786,232 | 264,057 |
| 6.25 | 3,803,743 | 2,015,821 | 1,520,725 | 267,197 | |

### Example 5: Ferritin Quantitation in the Pea Protein Extract

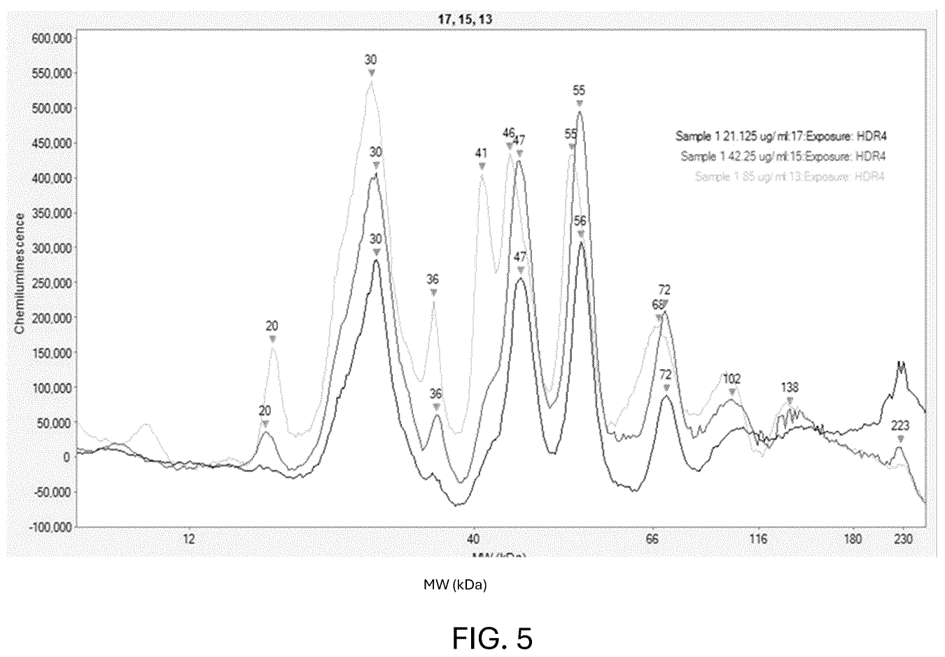

Ferritin was extracted from peas using the methods described above. Sample of starting pea concentrate was hydrolyzed for a minimum of 4 hours in a shaker table with water. Samples were spun in an ultra-centrifuge for 10 minutes at 35,000 G. For the second extraction the previously spun down sample was again re-hydrolyzed for a minimum of 2 hours and spun down again in similar manner. Two extractions were done. The WES system results from the first extraction are shown in FIG. 5, which shows the first water extraction of the starting pea protein concentrate at decreasing dilutions. The monomer, dimer, and trimers can be seen.

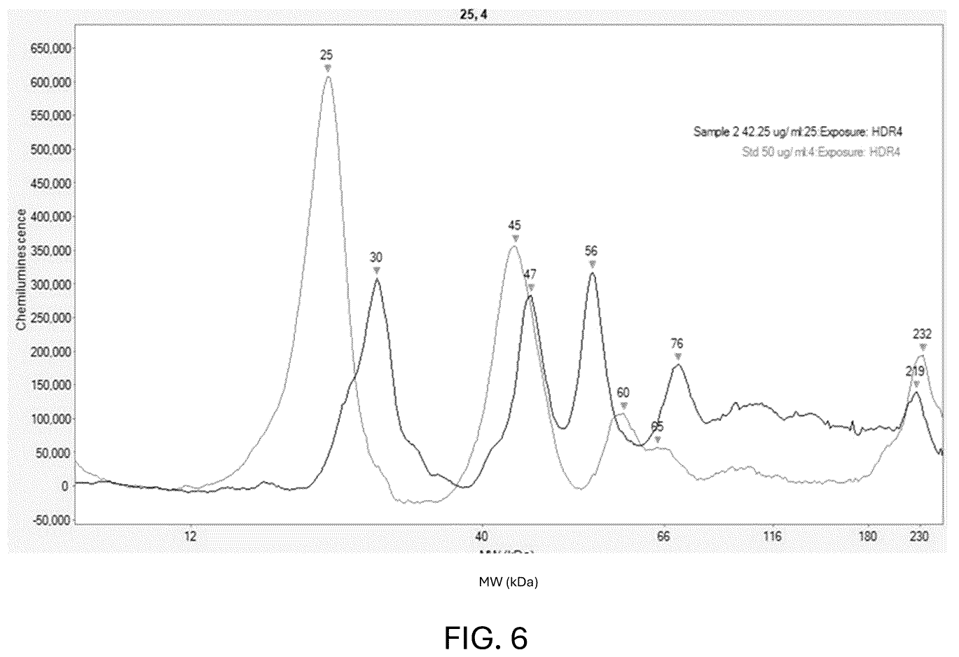

The results from the second extraction (compared to horse ferritin) are shown in FIG. 6. The data shows WES system results comparing 42.25 ug/ml of concentrated pea protein from the second extraction and 50 ug/ml horse ferritin standard.

The concentration of the ferritin from the starting pea protein can be determined from the WES system results. 240 grams total of pea protein extract was obtained. The initial sample was made at 16.7% solids, which is 40.08 g of pea protein extract as the starting solids mass. As an example, a dilution of 85 ug/ml would mean 85 μg/1,000 μg buffer or 1,000/85=11.76 times dilution.

From the first extraction, the calculation of 595 ug/ml ferritin in the original sample (50.6 ug/ml×11.76 (dilution)) was obtained as measured with the anti-ferritin antibody obtained in Example 2. After the second extraction, the calculation of 765.6 ug/ml of ferritin in the original sample (44.1 ug/ml×11.76 (dilution)) was obtained.

When the sample from the first extraction was assayed again using a newly produced antibody as described in Example 2, 1,862 g/ml ferritin was calculated to be in the original sample (158.3 ug/ml×11.76 (dilution)). 131 ml of pea protein extract resulted in 243,871 μg (or 243.871 mg) of ferritin. This resulted in a yield of 0.610% ferritin (243.871 mg Ferritin/40.08 g pea protein×100%). The extracted sample was concentrated in a convection air dryer by drying down the sample by 5.03% or 100%/5.03%=19.88 times more concentrated. This resulted in a calculation of 12.13% (0.610%×19.88) of starting ferritin.

After the second extraction, the calculation of 518 ug/ml of ferritin in the original sample (65.1 ug/ml×11.76 (dilution)) was obtained using the newly produced antibody. When the sample from the second extraction was assayed again using a newly produced antibody as described in Example 2, 765.6 ug/ml ferritin was calculated to be in the original sample (65 ug/ml×11.76 (dilution)). 128 ml of pea protein extract resulted in 97,997 μg (or 97.997 mg) of ferritin. The extracted sample was concentrated as described above by drying down the sample resulting in 22% of ferritin in the combined first and second pea protein extracts.

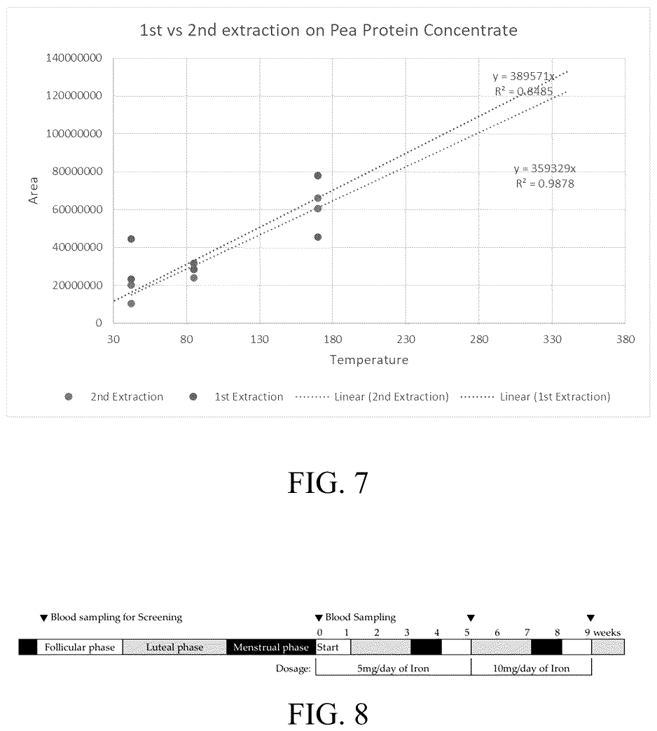

FIG. 7 shows the WES system data from the first and the second water extraction. The two extractions have similar amounts of ferritin present.

Table 6 depicts the actual measured values of ferritin in starting pea protein concentrated extracts fit to the known standard curve of horse ferritin.

TABLE 6
Ferritin concentration from pea extracts

| | Pea | |
|---|---|---|
| Extraction | Concentration (ug/ml) | Ferritin ug/ml |
| 1 | 340 | |
| 1 | 340 | |
| 1 | 170 | 76.18 |
| 1 | 170 | 130.62 |
| 1 | 85 | 47.99 |
| 1 | 85 | 53.14 |
| 1 | 42.25 | 74.85 |
| 1 | 42.25 | 39.13 |
| 1 | 21.125 | 56.20 |
| 1 | 21.125 | 19.60 |
| 2 | 340 | |
| 2 | 340 | |
| 2 | 170 | 110.73 |
| 2 | 170 | 101.54 |
| 2 | 85 | 40.34 |
| 2 | 85 | 47.79 |
| 2 | 42.25 | 17.17 |
| 2 | 42.25 | 33.94 |

TABLE 7
Calculation for the percent of solids in the first, second
and third pea protein concentrate starting material

| | Sample | Tare | Filled | Solution | Dry | Product | Precent | |
|---|---|---|---|---|---|---|---|---|
| | Number | Weight | Weight | Weight | Weight | Weight | Solids | Average |
| 1st | 1 | 7.6 | 45.2 | 37.6 | 9.5 | 1.9 | 5.05% | 5.03% |
| Extract | 2 | 7.6 | 43.6 | 36 | 9.4 | 1.8 | 5.00% | |
| | 3 | 7.6 | 51.4 | 43.8 | 9.8 | 2.2 | 5.02% | |
| 2nd | 4 | 7.7 | 54.9 | 47.2 | 8.3 | 0.6 | 1.27% | 1.11% |
| Extract | 5 | 7.7 | 53.6 | 45.9 | 8.2 | 0.5 | 1.09% | |
| | 6 | 7.7 | 38.7 | 31 | 8 | 0.3 | 0.97% | |
| 3rd | 7 | 8.5 | 48.1 | 39.6 | 8.5 | 0 | 0.00% | −0.09% |
| Extract | 8 | 8.6 | 65.8 | 57.2 | 8.5 | −0.1 | −0.17% | |

### Example 6—Toxicity Study/Safety Data of Ferritin in Rats

A repeated dose 90-days oral toxicity and safety study of ferritin was performed in Sprague Dawley rats. This study was designed to provide information on the effects of repeated exposure of ferritin, to establish No Observed Adverse Effect Level (NOAEL), to provide information on reversibility, delayed toxicity, selection of concentration for longer term studies and target organ toxicity of the ferritin test item in Sprague Dawley rats.

120 Rats (60 Male and 60 Female) were divided into 8 groups [G1-Vehicle (0 mg/kg b.wt. per day), G2—Low Dose (650 mg/kg b.wt. per day), G3—Mid Dose (1300 mg/kg b.wt. per day), G4—High Dose (1950 mg/kg b.wt. per day), G5—Vehicle Control Recovery (0 mg/kg b.wt. per day), G6—Low Dose Recovery (650 mg/kg b.wt. per day), G7—Mid Dose Recovery (1300 mg/kg b.wt. per day), G8—High Dose Recovery (1950 mg/kg b.wt. per day)]; 10 male+10 female in main group and 5 male and 5 female in recovery group animals per group.

Reverse Osmosis (R.O.) water was selected as a vehicle based on solubility testing. Required amount of the ferritin test item was received from the Test Item Control Office (TICO) for each concentration and dose groups separately and were formulated with R.O. water for low dose, mid dose, high dose, low dose recovery, mid dose recovery and high dose recovery groups. Samples of dose formulation were analyzed for homogeneity and dose concentration analysis once before commencement of dosing on day 1 of dosing and twice thereafter at monthly intervals during the treatment period. Samples were collected from lower, middle and top layers of the formulation prepared in air tight containers for homogeneity for all dose group levels and dose concentration analysis. Triplicate samples were collected each from lower, middle and top layers of each dose concentration of dose formulations including vehicle control (total 12 samples) for homogeneity and active ingredient concentration analysis. Dose formulation analysis was performed using validated analytical method. Test item formulation was administered orally by using 16 gauge oral gavage cannula attached with a syringe in Sprague Dawley rats for 90 days, consecutively. Animals from G2 (low dose), G3 (mid dose), G4 (high dose), G6 (low dose recovery), G7 (mid dose recovery) and G8 (high dose recovery) received the test item at a dose of 650, 1300 and 1950 mg/kg b.wt. per day respectively. Animals from G1 (vehicle) and G5 (vehicle control recovery) received R.O. water alone. A dose volume of 10 mL/kg b.wt. was maintained for all control and dose treated group animals.

All animals were observed for mortality/morbidity check twice in a day until terminal sacrifice with cage side observation of main and recovery group animals until day 91 and 118 respectively. The body weight of all animals from all 4 main groups and 4 recovery groups were measured once prior to treatment and weekly thereafter until terminal sacrifice. All animals from each group were observed for detailed clinical observations including discharge, locomotor activity, skin, mucous membrane, eyes, ears, oral cavity, fur, respiration, urine/feces, etc. once before treatment and weekly thereafter until terminal sacrifice. Feed consumption was measured after post treatment on weekly basis. All animals from the dose groups and vehicle group survived up to scheduled termination.

No mortality/morbidity was observed in any of the treated animals throughout the study period. No abnormal clinical signs were observed in any of the animals throughout the study period. No test item related significant changes were observed in body weight and feed consumption in the treated groups as compared to concurrent control group. No abnormal changes were observed in the detailed clinical examination prior to treatment and weekly during the treatment and ophthalmoscopy examination. All clinical pathology parameters in test item treated groups were comparable with the control group. No test item related significant changes were observed in clinical pathology parameters in treated group compared to control group in both sexes. No test item related significant changes were observed in absolute and relative organ weight in both sexes when compared to respective control group. No test item related findings were observed in any of the organ during gross (macroscopic) and microscopic observation.

Based on the results obtained, it could be concluded that the tested isolated ferritin of the invention did not produce any systemic toxicity or adverse effects up to the highest dose level (1950 mg/kg b.wt. per day) when administered orally for 90 consecutive days, under the conditions and procedures followed in the present study. Additionally, based on the results obtained from recovery group, it could be concluded that the test item did not produce any systemic toxicity or reversibility or delayed toxicity at highest dose level (1950 mg/kg b.wt. per day). Based on the findings, the No Observed Adverse Effect Level (NOAEL) of the tested ferritin was determined to be 1950 mg/kg b.wt. per day in Sprague Dawley rats. The tested ferritin is 5% iron, so the dose used in rats translates to 97.5 mg/kg of body weight of iron. (1950 mg/kg×5%=97.5 mg). The results can be extrapolated to humans. Since the average weight of a human male is 70 kg, 70 kg×97.5 mg/kg of Fe=6,825 mg/70 kg human, or 6.825 grams of Fe per 70 kg human daily for 90 days with no adverse events.

### Example 7: Nutritional Profile of Isolated Ferritin

A typical nutritional profile from 100 grams of the isolated ferritin of the invention is shown in Table 8.

TABLE 8
Ferritin nutritional profile

| | |
|---|---|
| Unit | Value |

| | | | |
|---|---|---|---|
| Calories | Kcal/100 | g | 311.8 |
| Calories from fat | Kcal/100 | g | 42.84 |
| Total Fat | g/100 | g | 4.76 |
| Saturated Fat | g/100 | g | 1.25 |
| Protein | g/100 | g | 66.92 |
| Cholesterol | mg/100 | g | <2 |
| Sodium | mg/100 | g | 3,276.43 |
| Calcium | mg/100 | g | 126.81 |
| Iron | mg/100 | g | 5336.09 |
| Carbohydrates | g/100 | g | 0.37 |
| Sugar | g/100 | g | 0.36 |
| Total Dietary Fiber | g/100 | g | 15.33 |
| Potassium | mg/100 | g | 158.22 |
| Moisture | g/100 | g | 5.37 |

### Example 8: Restorative Effect of Iron from Bean Ferritin on Low Hemoglobin 2 Level Caused by Menstruation in Japanese Women: a Randomized, Double-Blind Placebo-Controlled Intergroup Trial

Recently, bean ferritin has been attracting attention as a source of iron which is also available to vegetarians. Although high rates of iron absorption and bioavailability from this protein have been reported, the clinical data on its efficacy are still scarce. In this study, bean ferritin iron was administered to premenopausal Japanese women for nine weeks starting immediately after menstruation in order to evaluate their recovery from low hemoglobin level as one sign of anemia. Subjects in the test supplement group received an iron intake of 5 mg from one capsule containing bean extract (containing the isolated ferritin of the invention) for five weeks, which was increased to 10 mg (i.e., two capsules) from the 6th to 9th week. The study evaluated the change in hemoglobin levels as the primary endpoint, and hematocrit, red blood cell count, serum iron, mean corpuscular hemoglobin (MCH), mean corpuscular hemoglobin concentration (MCHC), serum ferritin, TSAT (TIBC), serum zinc, serum copper, anemia symptoms questionnaire, OSA sleep inventory, and the anti-fatigue questionnaire as the secondary endpoints. The results showed a significant difference (P=0.03) in the change in hemoglobin levels between the groups after nine weeks of intake, confirming the restorative effect of bean ferritin on low hemoglobin level caused by menstruation. Moreover, a significant difference (P=0.01) was observed in the amount of change in MCHC between the two groups after five weeks of intake, and after nine weeks of intake, a significant difference in the change in both MCH (P=0.02) and MCHC (P<0.01) was observed between the groups. A significant difference (P=0.03) was observed in the change in the serum ferritin levels after nine weeks of intake. The study confirmed that iron supplementation from bean ferritin containing the isolated ferritin of the invention is an effective treatment for low hemoglobin level and low ferritin level caused by menstruation.

Iron is an important component of a healthy diet, especially in menstruating women. The recommended daily intake for iron in menstruating adult women in Japan is 10.5 mg. A survey conducted by the Japanese Ministry of Health, Labour and Welfare revealed that the daily intake of iron in Japanese adult women was 7.2 mg, which is approximately 70% of the recommended value, indicating a high prevalence of iron deficiency among Japanese adult women. It has been estimated that the prevalence of various types of iron deficiency from the results of a survey of 3,015 Japanese women were: iron-deficiency anemia: 8.5%; latent iron deficiency: 8.0%; storage iron deficiency: 33.4%; normal: 43.6%; and other: 6.5%. The study showed that half of the women who participated in the survey were iron deficient or potentially iron deficient. In this study, the criteria for diagnosing anemia were determined from the Hb levels derived from the average values of healthy adults (i.e., transferrin saturation ≥16%, and serum ferritin levels ≥12 ng/ml). One of the reasons for the high prevalence of iron deficiency among Japanese adult women may be that the recommended daily intake for iron in Japan is considerably lower than the reference iron intake in the U.S. (18 mg/day).

As a result of this iron deficiency, several Japanese women are commonly prescribed iron preparations and iron supplements. Iron preparations contain ferrous sulfate and ferric citrate, while iron supplements contain ferric citrate, iron pyrophosphate, and heme iron. Ferritin, the most important iron-storing protein, was isolated from soybean seeds in 1987. In recent years, following a variety of basic studies, properties, such as iron absorption kinetics and bioavailability, as well as the practical evaluation of ferritin as a dietary iron source have also been explored. Because the iron absorption capacity and bioavailability from ferritin are like those from ferrous sulfate, a highly absorbable iron preparation used in pharmaceutical products, the protein was marketed as a new highly absorbable iron supplement. While most studies to date have been carried out on laboratory samples, human clinical trials using widely available iron sources have not yet been conducted.

Soybean- and pea-derived ferritin iron are on the market. Compared to the iron sulfate used in pharmaceuticals, ferritin iron has few adverse effects, including extremely low iron taste and gastrointestinal disorders. Moreover, because of its high absorbency despite being a non-heme iron, ferritin iron can be ingested by vegetarians and vegans.

According to the Dietary Reference Intakes for Japanese issued by the Ministry of Health, Labour and Welfare, blood iron loss during menstruation is estimated to be 3.06 mg/day for women aged 10-17 years, and 3.64 mg/day for those aged 18 years or older. Additionally, a decrease of 30% or more in ferritin levels during menstruation was reported. The severity of iron deficiency in adult menstruating women may vary depending on the timing of their menstrual cycle. For this reason, it is worth noting that an accurate evaluation in adult menstruating women who receive oral iron supplements is only possible if blood sampling is carried out within a set amount of time from the end of the menstrual cycle. Therefore, careful management of subjects is required.

In this study, the administration of trial supplements and hematologic evaluation in healthy premenopausal women who experienced anemia symptoms by low hemoglobin levels were performed at the same time during their menstrual cycle, while controlling for menstrual cycle phases, to assess the effects of bean iron from test supplements on low hemoglobin level caused by menstruation. Consumption of trial supplements was started within one week from the end of menstruation. Blood samples were collected after five and nine weeks from the late follicular to the luteal phase of the menstrual cycle to assess the restorative effect of bean ferritin (containing the isolated ferritin of the invention) on low hemoglobin level caused by menstruation. The amount of iron in the test supplement was increased from 5 mg/day to 10 mg/day after five weeks of intake, and the dose dependency was also evaluated.

Methods. Trial supplements. The participants of the study were divided into two groups. One group received a test supplement containing bean ferritin (the isolated ferritin of the invention) and the other group received a placebo control supplement. The bean ferritin in the test supplement used in the study contains approximately 5% iron. By contrast, the control supplement did not contain any iron. Both the test and control supplements were packed as porcine gelatin capsules with a content weight of 280 mg per capsule and colored white with titanium dioxide. Corn starch was used as a basic ingredient for both trial supplements. The test supplement contained 5 mg of iron per capsule (100 mg of bean extract), while in the control supplement, the bean extract was replaced with an equal amount of corn starch.

Subjects. The subjects were recruited among paid volunteers based on the inclusion and exclusion criteria outlined below, and 40 out of 80 women who gave their consent to participate in the study were selected based on the laboratory values of hemoglobin at screening and the inclusion and exclusion criteria described below. The target number of subjects was determined based on previous studies. The subjects were randomly assigned to two groups, through stratified randomization. The groups were kept secret until the end of the study to ensure the blinding of participants, intervention providers, and outcome assessors. The study was conducted in accordance with the CONSORT 2010 Statement (Consolidated Standards of Reporting Trials 2010 Statement) for reporting randomized controlled trials. In addition, an outline of the study was registered in the public database UMIN Clinical Trials Registry (ID: UMIN000045253).

After the completion of the study, the allocation order and numbering of the participants were disclosed, and the groups of subjects that received the bean extract and the control supplement were designated as S group and P group, respectively. Before starting the screening process, the subjects were fully informed on content and methods of the study and their written consent was obtained. The ethical, scientific, and clinical validity of the study was reviewed and approved by the Institutional Review Board of Tsukuji Futaba Clinic, Hikobae-kai Medical Corporation. The study was conducted in accordance with the ethical principles of the Declaration of Helsinki and the Ethical Guidelines for Medical and Health Research Involving Human Subjects.

Inclusion criteria were as follows: 1) women who experienced symptoms of low hemoglobin level (anemia) on a daily basis, 2) premenopausal women, 3) BMI of less than 30 kg/m², 4) menstrual cycle timing falls within the following time frames: the timing of the menstrual cycle is for those who fall within the specified time period, 5) with a relatively stable menstrual cycle, 6) provided written consent to participate in the study, and 7) Hb levels of less than 13 g/dL on a previous blood test.

Exclusion criteria were as follows: 1) women who are allergic to beans (e.g., soybean and pea), 2) currently on medication for iron-deficiency anemia and receiving medication for any disease, 3) consume food (e.g., soy milk) and health food products containing the ingredients of the test supplement used in the study, 4) irregular menstrual cycles, 5) serious diseases affecting the glycometabolism, lipid metabolism, liver function, kidney function, heart, circulatory system, respiratory system, endocrine system, immune system, and nervous system, or mental disorders, and women with a history of such diseases, 6) medication for a disease or with a history of a serious disease requiring medication, 7) develop allergies related to the study, 8) participating in other clinical research at the time of initiation of the study, 9) pregnant or planning to get pregnant or breastfeed during the study period, 10) uterine myomas or endometriosis, and 11) judged by the principal investigator to be unsuitable to participate in the study.

Study design and methods. The study, which used two kinds of supplements, was designed as a randomized double-blind placebo-controlled intergroup trial and was carried out at Hasegawa Clinics, Seishukai Medical Corporation, on two groups of women selected based on the timing of their menstrual cycle. The study schedule and menstrual cycle of the subjects are shown in FIG. 8.

Both trial supplements were distributed to the subjects at the time of arrival to maintain blindness and were administered as follows: one capsule per day (5 mg of iron/day), preferably taken at breakfast with water or lukewarm water on an empty stomach. The subjects received the supplements for nine weeks; however, from the end of the 5th week the dose was doubled to two capsules per day (10 mg of iron/day).

The reason for increasing the dosage was to verify the difference between the 5 mg/day dosage and the 10 mg/day dosage. The 5 mg/day dosage was intended to supplement the approximately 4.1 mg/day dosage that Japanese adult women lack, while the 10 mg/day dosage was intended to supplement the recommended intake for adult women in Japan. During the period of intake of trial supplements, the subjects were asked to record daily their menstrual cycle phases, living conditions, dietary composition, and whether they took medications or the trial supplements. Subsequently, the subjects were asked to visit the hospital before, and at five, and nine weeks after intake, or upon discontinuation, and as may be necessary, and were then asked to complete a web-based questionnaire on the anemia symptoms before, and at five, and nine weeks after the intake of trial supplements. Next, blood samples were collected to evaluate the efficacy and gather observational items (e.g., adverse events, body weight, BMI, blood pressure, pulse, and biochemical and hematological tests) before, and at five, and nine weeks after intake. In addition, biochemical and hematological tests were also performed.

Efficacy endpoints. In this study, the amount of change in the Hb levels was used as a primary endpoint for screening and efficacy evaluation. In addition to hematocrit, the red blood cell count, serum iron, MCH, MCHC, serum ferritin, and TSAT (TIBC), which are common indicators of iron deficiency, serum zinc and copper were also measured as secondary endpoints to evaluate the zinc and copper levels in the blood as the absorption of these two elements may be antagonized by iron intake. Additionally, questionnaire surveys on anemia symptoms, OSA sleep, and anti-fatigue were conducted to evaluate changes in quality of life. In addition to statistical analysis, the mean, standard deviation, standard error, median, and minimum and maximum values were calculated for all endpoints.

Statistical analysis. Statistical analysis for efficacy evaluation was carried out by Kansai University of Welfare Sciences. Firstly, an intra-group comparison in the study population was performed before, and at five, and nine weeks after intake. Since subjects varied in age and severity of iron deficiency, differences in each endpoint were already observed at the pre-consumption stage. In order to evaluate the efficacy of the treatment, while also taking into account this variation, the amount of change before, and at five, and nine weeks after intake was also calculated. Next, an intergroup comparison was performed. Intragroup and intergroup comparisons were analyzed using a paired t-test, and Student's t-test, respectively. The tests were applied assuming the normality of each dataset (while multiplicity was not considered) with a significance level of 5% (two-tailed test). SAS9.4 (SAS Inc.) was used for statistical analysis.

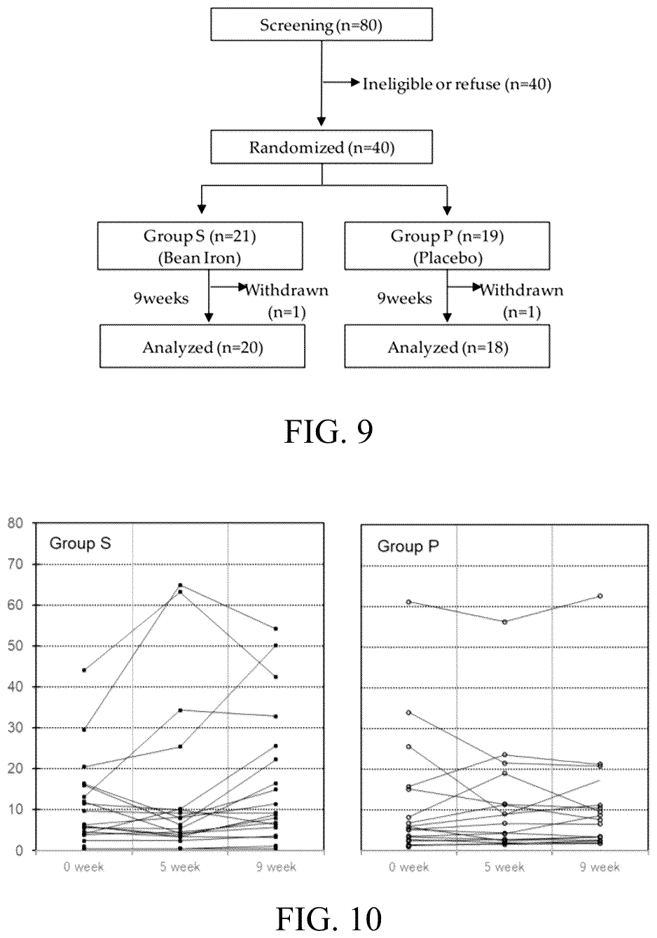

Results. A flow diagram of the progress from screening to analysis is shown in FIG. 9. Two subjects who violated the exclusion criteria after inclusion were excluded from the analysis. The baseline characteristics of the subjects are shown in Table 9, and no significant differences between the two groups in terms of age, BMI, hemoglobin, ferritin were noted. In Table 10, the subjects were classified based on the six degrees of severity of iron deficiency. No bias was observed in any of the two groups, and the composition of data in the subjects' low hemoglobin level was uniform.

Since the study also considers the timing of the menstrual cycle phases, Table 11 shows the number of days from the end of menstruation. In this case also, no substantial bias was observed in either group. However, despite controlling for menstrual cycle phases (i.e, from the late follicular through the luteal phase), after nine weeks, the time that elapsed from the end of menstruation to blood sampling in the S group became shorter than in the P group, making it difficult to assess recovery from low hemoglobin level. No subjects with menorrhagia were observed.

TABLE 9
Baseline characteristics of subjects and timing
of blood sampling in the S and P groups.

| Variable | Group P (n = 18) | Group S (n = 20) | p-value |
|---|---|---|---|
| Age (years) | 34.9 ± 9.3 | 32.4 ± 9.4 | 0.3995 |
| BMI (kg/m²) | 20.6 ± 2.7 | 19.4 ± 2.5 | 0.1596 |
| Hb (g/dL) | 11.4 ± 1.7 | 11.2 ± 1.9 | 0.7623 |
| RBC (/μL) | 412.7 ± 37.8 | 417.8 ± 27.4 | 0.6324 |
| Ht (%) | 37.3 ± 4.4 | 37.4 ± 4.7 | 0.9917 |
| Ferritin (ng/ml) | 18.6 ± 31.8 | 15.0 ± 19.9 | 0.6705 |
| TSAT (%) | 13.4 ± 7.8 | 15.2 ± 0.3 | 0.5482 |

Mean ± SD

TABLE 10
Classification of iron deficiency

| | Group P (n = 18) | Group S (n = 20) |
|---|---|---|
| Iron deficiency anemia | 8 | 9 |
| Latent iron deficiency | 2 | 3 |
| Pre-latent iron deficiency | 4 | 4 |
| Low TSAT | 2 | 1 |
| Non iron deficiency | 0 | 3 |
| Others | 2 | 0 |

TABLE 11
Number of days from end of menstruation to blood sampling

| Sampling Data | | | Group P (n = 18) | Group S (n = 20) |
|---|---|---|---|---|
| 0 weeks | 0 | days | 0 | 0 |
| | 1-7 | days | 0 | 0 |
| | 8-14 | days | 0 | 0 |
| | 14- | days | 18 | 20 |
| 5 weeks | 0 | days | 0 | 2 |
| | 1-7 | days | 3 | 2 |
| | 8-14 | days | 7 | 10 |
| | 14-21 | days | 9 | 7 |
| 9 weeks | 0 | days | 2 | 0 |
| | 1-7 | days | 9 | 5 |
| | 8-14 | days | 4 | 8 |
| | 14-21 | days | 4 | 8 |

*Parts of 3 blood sampling (0, 5 and 9 week) could not be conducted in 2 subjects due to infection with COVID-19.
These subjects were removed from the evaluation.

Evaluation of efficacy by blood test. Table 12 shows the values of primary and secondary endpoints before (0 week), and at five, and nine weeks after intake as well as the amount of change from before, to five and nine weeks after intake.

The amount of change in the primary endpoint of Hb levels showed no significant intergroup differences after five weeks of intake; however, significant intergroup differences were observed after nine weeks of intake. Among the secondary endpoints (e.g., hematocrit, red blood cell count, serum iron, MCH, MCHC, serum ferritin, TSAT, serum copper, and serum zinc), a significant difference (P=0.01) in the amount of change in the MCHC levels between groups was observed after five weeks of intake, and a significant difference in the amount of change in MCH (P=0.02) and MCHC (P<0.01) levels between groups was observed after nine weeks of intake. The other items showed significant intragroup differences (e.g., red blood cell count and MCV); however, no significant intergroup differences were observed.

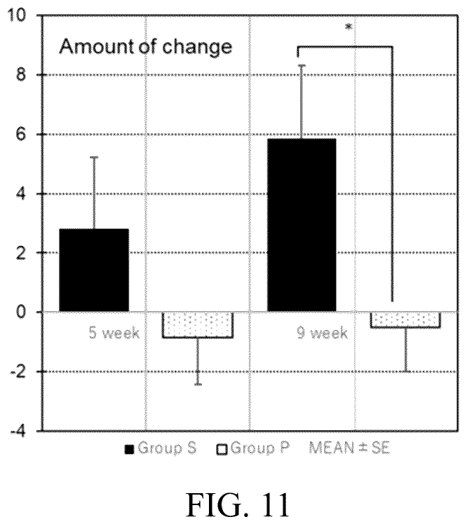

FIG. 10 shows changes in the serum ferritin levels for each subject before and after intake of trial supplements, and FIG. 11 shows the amount of change after five and nine weeks of intake. A large number of subjects in the S group showed an increase in ferritin levels, indicating a significant difference between the two groups after nine weeks of intake.

TABLE 12
Primary and secondary outcomes of subjects in the S and P groups.

| | | | | |
|---|---|---|---|---|
| S group | P-Value | P group | P-Value | P-Value |
| MEAN ± SD | in Change | MEAN ± SD | in Change | S vs P |

Primary outcome

| | | | | | | |
|---|---|---|---|---|---|---|
| Hb | 0 w | 11.2 ± 1.8 | | 11.4 ± 1.6 | | 0.78 |
| (g/dL) | 5 w | 11.3 ± 1.8 | | 11.5 ± 1.6 | | 0.68 |
| | 9 w | 11.8 ± 1.9 | | 11.4 ± 1.6 | | 0.53 |
| | 5 w-0 w | 0.1 ± 0.6 | 0.55 | 0.1 ± 0.5 | 0.30 | 0.75 |
| | 9 w-0 w | 0.6 ± 0.8 | <0.01 | 0.1 ± 0.7 | 0.71 | 0.03 |

Secondary outcomes

| | | | | | | |
|---|---|---|---|---|---|---|
| RBC | 0 w | 420.3 ± 29.1 | | 414.4 ± 37.5 | | 0.58 |
| (/μL) | 5 w | 422.3 ± 26.3 | | 420.9 ± 32.3 | | 0.88 |
| | 9 w | 433.1 ± 30.3 | | 418.6 ± 31.6 | | 0.16 |
| | 5 w-0 w | 2.0 ± 22.6 | 0.68 | 8.2 ± 20.6 | 0.11 | 0.38 |
| | 9 w-0 w | 15.3 ± 23.2 | <0.01 | 5.9 ± 26.0 | 0.35 | 0.25 |
| Ht | 0 w | 37.4 ± 4.5 | | 37.4 ± 4.3 | | 0.98 |
| (%) | 5 w | 36.8 ± 4.3 | | 37.7 ± 4.2 | | 0.52 |
| | 9 w | 38.2 ± 4.6 | | 37.5 ± 4.3 | | 0.60 |
| | 5 w-0 w | −0.6 ± 1.9 | 0.18 | 0.3 ± 2.2 | 0.54 | 0.18 |
| | 9 w-0 w | 0.9 ± 2.2 | 0.09 | 0.1 ± 2.3 | 0.82 | 0.31 |
| MCV | 0 w | 89.0 ± 9.7 | | 90.2 ± 5.9 | | 0.64 |
| (fL) | 5 w | 87.0 ± 8.9 | | 89.4 ± 6.2 | | 0.35 |
| | 9 w | 88.3 ± 9.5 | | 89.4 ± 6.8 | | 0.67 |
| | 5 w-0 w | −1.9 ± 1.8 | <0.01 | −1.0 ± 2.4 | 0.10 | 0.19 |
| | 9 w-0 w | −1.1 ± 1.9 | 0.02 | −0.9 ± 2.2 | 0.09 | 0.82 |
| MCH | 0 w | 26.6 ± 4.0 | | 27.4 ± 2.7 | 0.00 | 0.51 |
| (pg) | 5 w | 26.7 ± 4.0 | | 27.3 ± 2.9 | 0.00 | 0.61 |
| | 9 w | 27.2 ± 4.1 | | 27.3 ± 2.9 | 0.00 | 0.98 |
| | 5 w-0 w | 0.1 ± 0.5 | 0.55 | −0.2 ± 0.5 | 0.07 | 0.08 |
| | 9 w-0 w | 0.5 ± 1.0 | 0.05 | −0.2 ± 0.8 | 0.21 | 0.02 |
| MCHC | 0 w | 29.8 ± 1.7 | | 30.3 ± 1.4 | | 0.35 |
| (%) | 5 w | 30.5 ± 1.8 | | 30.5 ± 1.3 | | 0.92 |
| | 9 w | 30.7 ± 1.9 | | 30.4 ± 1.1 | | 0.60 |
| | 5 w-0 w | 0.7 ± 0.6 | <0.01 | 0.1 ± 0.8 | 0.56 | 0.01 |
| | 9 w-0 w | 0.9 ± 0.7 | <0.01 | 0.1 ± 0.7 | 0.76 | <0.01 |
| Ferr | 0 w | 14.4 ± 19.6 | | 19.0 ± 31.0 | | 0.57 |
| (ng/ml) | 5 w | 15.9 ± 21.4 | | 21.9 ± 47.6 | | 0.60 |
| | 9 w | 18.8 ± 17.9 | | 18.5 ± 33.0 | | 0.98 |
| | 5 w-0 w | 1.5 ± 12.1 | 0.58 | 3.3 ± 18.7 | 0.95 | 0.72 |
| | 9 w-0 w | 3.8 ± 13.9 | 0.23 | −0.1 ± 6.2 | 0.34 | 0.28 |
| TSAT | 0 w | 15.1 ± 10.0 | | 13.5 ± 7.6 | | 0.57 |
| (%) | 5 w | 16.8 ± 11.1 | | 15.5 ± 9.9 | | 0.71 |
| | 9 w | 17.1 ± 10.2 | | 15.4 ± 10.6 | | 0.62 |
| | 5 w-0 w | 1.7 ± 7.0 | 0.29 | 2.1 ± 5.9 | 0.32 | 0.83 |
| | 9 w-0 w | 1.8 ± 8.0 | 0.32 | 2.0 ± 8.3 | 0.14 | 0.96 |
| Serum | 0 w | 53.4 ± 30.1 | | 50.1 ± 26.1 | | 0.72 |
| Iron | 5 w | 60.9 ± 33.5 | | 61.4 ± 36.5 | | 0.97 |
| (μg/dL) | 9 w | 64.9 ± 39.9 | | 62.8 ± 43.0 | | 0.88 |
| | 5 w-0 w | 7.5 ± 26.6 | 0.21 | 11.2 ± 23.1 | 0.00 | 0.65 |
| | 9 w-0 w | 11.8 ± 32.9 | 0.12 | 12.6 ± 36.1 | 0.16 | 0.94 |
| Serum | 0 w | 102.0 ± 9.9 | | 104.7 ± 23.9 | | 0.64 |
| Copper | 5 w | 105.1 ± 17.7 | | 111.9 ± 26.9 | | 0.35 |
| (μg/dL) | 9 w | 107.5 ± 15.2 | | 114.2 ± 23.4 | | 0.30 |
| | 5 w-0 w | 3.0 ± 14.3 | 0.34 | 6.4 ± 10.7 | 0.02 | 0.42 |
| | 9 w-0 w | 5.3 ± 14.8 | 0.13 | 8.6 ± 9.4 | <0.01 | 0.42 |
| Serum | 0 w | 93.9 ± 14.5 | | 95.4 ± 11.9 | | 0.72 |
| Zinc | 5 w | 90.7 ± 13.8 | | 85.4 ± 12.1 | | 0.97 |
| (μg/dL) | 9 w | 95.0 ± 12.5 | | 93.5 ± 10.4 | | 0.88 |
| | 5 w-0 w | −3.2 ± 12.7 | 0.26 | −10.1 ± 10.6 | <0.01 | 0.65 |
| | 9 w-0 w | 2.0 ± 13.2 | 0.51 | −2.0 ± 10.6 | 0.44 | 0.94 |

Various questionnaires. Significant intragroup differences in the amount of change in several items of the anemia symptoms questionnaire (e.g., subjective feeling of iron deficiency symptoms, sense of fatigue, sense of vertigo or dizziness, sleepiness, facial complexion, discomfort during exercise, unpleasant feelings of restlessness in the lower limbs, and taste perception) were observed. Notably, significant intragroup differences in such items, such as subjective feeling of iron deficiency symptoms, sense of fatigue, sense of vertigo or dizziness, and facial complexion were observed in both group S and P. Significant intergroup differences in taste perception were observed only after three and seven weeks of intake. No significant intergroup differences were observed in the OSA sleep inventory and anti-fatigue questionnaire (data not shown).

Safety and side effects. No adverse events were reported during the course of the study. In addition, no common side effects associated with iron supplementation (e.g., heartburn, nausea, abdominal pain, constipation, etc.) were reported. No significant differences were observed in such observational items as body composition, and biochemical and hematological tests, and changes for all the events were within acceptable values.

Discussion. The study showed significant intergroup differences in the primary endpoint of Hb levels, as well as in MCH and MCHC, which are used as a measure of red blood cell quality, and serum ferritin, an indicator of the body iron stores. Table 13 shows the change in iron anemia symptoms by low hemoglobin level before and after the consumption of trial supplements in subjects who were classified based on the six degrees of severity of iron deficiency. The results show that approximately half of the subjects in the S group had recovered from low hemoglobin level. Moreover, while some subjects in the P group recovered, although in a smaller number than in the S group, the number of subjects who deteriorated was significantly higher in the P than in the S group. These results confirm that the intake of iron from bean ferritin can effectively lead to recovery from low hemoglobin level or low ferritin level caused by menstruation.

TABLE 13
Change of iron deficiency from 0
week to 9 week in S and P groups

| | S group | | | P group | | |
|---|---|---|---|---|---|---|
| | 0 week | 5 week | 9 week | 0 week | 5 week | 9 week |
| Iron deficiency | 9 | 8 | 6 | 8 | 8 | 8 |
| anemia | | | | | | |
| Latent iron | 3 | 2 | 2 | 2 | 2 | 2 |
| deficiency | | | | | | |
| Pre-latent | 4 | 5 | 4 | 4 | 4 | 3 |
| iron | | | | | | |
| deficiency | | | | | | |
| Low TSAT | 1 | 1 | 1 | 2 | 1 | 0 |
| Non iron | 3 | 4 | 7 | 0 | 1 | 3 |
| deficiency | | | | | | |
| Others | 0 | 0 | 0 | 2 | 2 | 2 |
| Recovery | — | 7 | 6 | — | 2 | 4 |
| (vs 0 week) | | | | | | |
| Deterioration | — | 1 | 0 | — | 2 | 3 |
| (vs 0 week) | | | | | | |

The serum ferritin levels were slightly higher in the S group, which continued to receive bean iron at a dose of 5 mg/day for five weeks, but were not significantly different from those in the P group. After nine weeks of intake, on the other hand, there was a significant difference between the amount of change observed in the S group, which continued to receive bean iron at a dose of 10 mg/day for an additional four weeks (for a total of nine weeks), and that observed in the P group. At the same time, the serum ferritin levels of subjects in the S group increased approximately 1.6-fold on average compared to the beginning of the study, also suggesting an increase in the body iron stores.

Some studies reported that the serum ferritin levels, during menstruation, decrease by 30% or more, and, in particular, the follicular phase during and immediately after the end of menstruation is thought to be characterized by a decrease in ferritin levels. It has been reported that the main cause of iron deficiency in Japanese women is not insufficient intake, but iron loss through menstruation. Although iron stores are expected to be restored after menstruation, through dietary intake or other sources, it is worth noting that the majority of absorbed iron is transferred to the blood and used for hemoglobin formation. Consequently, body iron stores are believed to remain unchanged or even slightly reduced as shown by the changes observed in the P group in our study.

Moreover, in patients with iron-deficiency anemia treated with oral iron supplements, reticulocyte crisis (i.e., the rapid increase of the number of reticulocytes) occurs within 7-10 days of the start of treatment after an increase in serum iron levels, and is followed by an increase in Hb levels. Generally, it takes from three to four months (12 to 16 weeks) to fully replete iron stores (i.e., serum ferritin)). To restore the serum ferritin levels after menstruation and recover from iron-deficiency anemia or latent iron deficiency, iron should be supplied regularly from sources other than food. In case of iron supplements derived from beans, such as the one used in this study, a daily intake of 10 mg or more is recommended to ensure a more effective recovery. Moreover, the slight increase in the amount of change observed at a daily intake of approximately 5 mg, albeit not significant, suggests that continuous iron intake over a prolonged period (e.g., 12 weeks or longer) may lead to gradual recovery from low hemoglobin level.

In patients with low hemoglobin level, such as women during menstruation, elevated expression of iron homeostasis proteins, such as divalent metal transporter 1 (DMT1), Dcytb, ferroportin, and hephaestin is observed, and iron absorption from the digestive tract tends to increase. According to the National Health and Nutrition Survey conducted by the Ministry of Health, Labour and Welfare, approximately 70% of the iron consumed by Japanese from food sources is non-heme iron. It also should be noted that higher consumption of iron resulting from the concomitant intake of supplements as well as of food sources is known to inhibit the intestinal uptake of zinc and copper due to the competitive antagonism between these elements on the DMT1. In order to evaluate the inhibitory effect of iron from bean ferritin on copper and zinc uptake during iron absorption, in addition to the serum iron, serum zinc and copper was evaluated. The results showed no clear inhibitory trends, with no significant intergroup differences, although significant intragroup differences were observed in the P group.

Since both groups consumed iron from food sources on a daily basis, and the iron intake from test supplement in the S group was lower (5-10 mg) than that from commercially available supplements (prescription drugs supply an iron intake of 100-200 mg/day), the differences were not considered significant. In order to evaluate the inhibitory effect of iron from bean ferritin, an intake of 40 mg or more (i.e., the acceptable daily intake reported for Japanese women) would be necessary. In the anemia symptoms questionnaire, several items showed significant intragroup differences in both groups. Since these changes include the restorative effect of iron supplementation from dietary sources, the intragroup differences observed in both groups were considered to be significant. Moreover, while all P-values in the S group were smaller, indicating slightly stronger recovery trends, the difference between the groups was not large enough to be considered significant. Because responses to questionnaires are highly subjective and may easily reflect some placebo effect, statistically significant differences were difficult to derive due to the large number of subjects with no iron-deficiency anemia included in the analysis. According to the Nutritional and Dietary Guidelines for athletes issued by the Sports Medicine and Science Committee of the Japan Sports Association, the iron intake in athletes should be 1.5 times the recommended amount for general adult women (15 mg/day), considering iron loss due to sweating and intestinal bleeding caused by exercise.

As also mentioned above, the recommended iron intake for adult women in the U.S. is 18 mg/day. It might be preferable for women who are menstruating or immediately after menstruation, and iron-deficient women who had or experienced symptoms of iron deficiency, to consume iron at a dose of 15-18 mg/day, which is the recommended intake for athletes and adult women in the U.S. Since the average consumption of iron in adult Japanese women is 7.2 mg/day, supplementation with 8-10 mg/day of iron from sources other than food (e.g., supplements) would be advisable. These recommended values for iron-deficient women are also supported by the results of this study. In this study, subjects were recruited using hemoglobin as a primary endpoint, but after the end of the study, we found that some subjects who exhibited higher serum ferritin levels than the reference upper limit (80 ng/ml) and did not have primary anemia were erroneously included in the analysis. In future studies, reference values for serum ferritin levels should be specified in the selection/exclusion criteria to allow a more accurate evaluation of the restorative effects of iron intake in iron-deficient patients.

Conclusion. The significant difference in the amount of change in Hb levels observed in the study after nine weeks of intake of iron from bean ferritin confirmed that recovery from low hemoglobin level or low ferritin level caused by menstruation in Japanese women was possible. Also, an intake of iron of 5 mg/day from bean ferritin for five weeks has been shown to improve the red blood cell quality (MCHC). Significant differences were also shown in the change in ferritin levels after nine weeks of intake, suggesting that iron consumed at a dose of 10 mg/day is more likely to improve the low hemoglobin level and increase the body iron stores than at a dose of 5 mg/day. Therefore, pea-derived bean ferritin intake is effective against anemia.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure. All references cited herein are incorporated by reference in their entirety.

Insofar as the description above discloses any additional subject matter that is not within the scope of the claims below, the inventions are not dedicated to the public and the right to file one or more applications to claim such additional inventions is reserved.

What is claimed is:

1. A method for quantitatively determining the concentration of free iron and ferritin-bound iron in a sample, comprising:
(a) diluting a ferritin-containing sample with a diluent to create a slurry;
(b) processing the slurry through a crossflow filtration system equipped with a membrane of at least 500 kilodaltons (kDa) molecular weight cutoff;
(c) collecting the permeate containing free iron and the retentate containing ferritin-bound iron;
(d) analyzing the iron concentrations in the permeate and retentate using a suitable analytical technique; and
(e) calculating the mass balance of the total iron content.

2. The method of claim 1, wherein the ferritin-containing sample is diluted with water in a ratio of 1:5 to 1:10 to create the slurry.

3. The method of claim 1, wherein the filtration is conducted at a backpressure of 15 psi and a flow rate of at least 1,500 mL/hr.

4. The method of claim 1, wherein the iron concentration in the permeate and retentate is analyzed using atomic absorption spectroscopy (AAS) or inductively coupled plasma spectroscopy (ICP).

5. The method of claim 1, further comprising monitoring the permeate flow rate using a scale under the sample tank to detect membrane fouling.

6. The method of claim 1, wherein the filtration system includes an automatic flushing mechanism to reduce membrane fouling.

7. The method of claim 1, wherein the analytical technique used for iron concentration analysis is colorimetric using a ferrozine-based assay.

8. The method of claim 1, further comprising pre-treating the ferritin-containing sample with a chelating agent to stabilize free iron during filtration.

9. The method of claim 1, wherein the slurry is maintained at a controlled temperature between 4° C. and 25° C. during filtration to preserve protein integrity.

10. The method of claim 1, further comprising the use of an ultrafiltration step prior to crossflow filtration to remove impurities smaller than 10 kDa.

11. A crossflow filtration system for separating free iron from ferritin-bound iron, comprising:
(a) a sample tank for holding a slurry of ferritin-containing material;
(b) a crossflow filter with a 500 kDa molecular weight cutoff membrane;
(c) a pump configured to recirculate the slurry through the filter at a flow rate of at least 1,500 mL/hr;
(d) a backpressure regulator to maintain a transmembrane pressure of 15 psi;
(e) a permeate receiving vessel for collecting free iron; and
(f) means for monitoring and analyzing the iron content in the permeate and retentate.

12. The system of claim 11, wherein the membrane is made of polysulfone, polyethersulfone, or another biocompatible material.

13. The crossflow filtration system of claim 11, wherein the system includes an integrated spectrophotometer for real-time analysis of iron concentrations in the permeate.

14. The crossflow filtration system of claim 11, wherein the sample tank is equipped with an agitator to prevent settling of ferritin-containing material.

15. The crossflow filtration system of claim 6, further comprising an automated system for continuous permeate and retentate collection.

16. The crossflow filtration system of claim 6, wherein the system includes a temperature regulation unit to maintain optimal processing conditions.

17. A kit for determining ferritin-bound iron in a sample, comprising:
(a) a crossflow filtration membrane with a molecular weight cutoff of at least 500 kDa;
(b) instructions for diluting a ferritin-containing sample and processing it through the filtration system;
(c) components for collecting permeate and retentate samples;
(d) reagents or tools for determining iron concentration in the permeate and retentate; and
(e) a guide for performing mass balance calculations to determine free and ferritin-bound iron.

18. The kit of claim 17, further comprising ferritin calibration standards derived from animal or plant sources.

19. The kit of claim 17, further comprising disposable filters for one-time use in laboratory or field applications.
